SEQUENCE LISTING (26 sequences)

SEQ ID NO: 1 - protein, 5 aa, Artificial Sequence
  CDR1
  MISC_FEATURE (1)..(1): X is F or Y
XTGVA

SEQ ID NO: 2 - protein, 9 aa, Artificial Sequence
  CDR2
  MISC_FEATURE (1)..(1): X is L, T or S
  MISC_FEATURE (2)..(2): X is I, T or A
  MISC_FEATURE (4)..(4): X is A or G
  MISC_FEATURE (7)..(7): X is A or G
  MISC_FEATURE (9)..(9): X is R or K
XXTXDGXTX

SEQ ID NO: 3 - protein, 5 aa, Artificial Sequence
  CDR3
  MISC_FEATURE (4)..(4): X is E or R
  MISC_FEATURE (5)..(5): X is Y or F
KTGXX

SEQ ID NO: 4 - protein, 22 aa, Human cytomegalovirus
  MISC_FEATURE (8)..(8): X is A or T
  MISC_FEATURE (15)..(15): X is D or E
  MISC_FEATURE (18)..(18): X is D, E or L
  MISC_FEATURE (19)..(19): X is E, D or G
MTPTTTTXELTTEFXYDXXATP

SEQ ID NO: 5 - protein, 5 aa, Artificial Sequence
  CDR1
  MISC_FEATURE (1)..(1): X is any amino acid
XTGVA

SEQ ID NO: 6 - protein, 9 aa, Artificial Sequence
  CDR2
  MISC_FEATURE (1)..(1): X is any amino acid
  MISC_FEATURE (2)..(2): X is any amino acid
  MISC_FEATURE (4)..(4): X is any amino acid
  MISC_FEATURE (7)..(7): X is any amino acid
  MISC_FEATURE (9)..(9): X is any amino acid
XXTXDGXTX

SEQ ID NO: 7 - protein, 5 aa, Artificial Sequence
  CDR3
  MISC_FEATURE (4)..(4): X is any amino acid
  MISC_FEATURE (5)..(5): X is any amino acid
KTGXX

SEQ ID NO: 8 - protein, 5 aa, Artificial Sequence
  CDR1
FTGVA

SEQ ID NO: 9 - protein, 9 aa, Artificial Sequence
  CDR2
LITGDGATR

SEQ ID NO: 10 - protein, 5 aa, Artificial Sequence
  CDR3
KTGEY

SEQ ID NO: 11 - protein, 6 aa, Artificial Sequence
  His-tag
HHHHHH

SEQ ID NO: 12 - protein, 10 aa, Artificial Sequence
  c-myc domain
EQKLISEEDL

SEQ ID NO: 13 - protein, 9 aa, Artificial Sequence
  hemagglutinin tag
YPYDVPDYA

SEQ ID NO: 14 - protein, 15 aa, Artificial Sequence
  Gly-Ser linker
GGGGSGGGGSGGGGS

SEQ ID NO: 15 - protein, 35 aa, Artificial Sequence
  Gly-Ser linker
GGGGSGGGGSGGGGSGGGGSGGGGSGGGGSGGGGS

SEQ ID NO: 16 - protein, 15 aa, Artificial Sequence
  Gly-Ser linker
GGGSSGGGSSGGGSS

SEQ ID NO: 17 - protein, 114 aa, Artificial Sequence
  VUN100 VHH
EVQLVESGGGLVQPGGSLRLACAVSGPGLIFKFTGVAWYRRQVPGAKRGLVALITGDGATRYGDSVKGRFTVSRDIAAKRVYLEMNDLRSEDTAVYYCKTGEYWGQGTQVTVSS

SEQ ID NO: 18 - protein, 111 aa, Artificial Sequence
  US28-Nb1 VHH
EVQLVESGGGLVQAGGSLRLSCVVSGTIFSYTGVAWYRQTSGKQREWVATTTNDGGTKFADSVKGRFTISRDNAKKTVYLQMNNLNAEDTAVYYCKTGRFWGRGTLVTVSS

SEQ ID NO: 19 - protein, 111 aa, Artificial Sequence
  US28-Nb2 VHH
EVQLVESGGGLVQAGGSLRLSCVVSGTIFSYTGVAWYRQTSGNQREWVATATNDGGTKFADSMKGRFTISRDNAKKTVYLQMNNLNAEDAAVYYCKTGRFWGRGTLVTVSS

SEQ ID NO: 20 - protein, 111 aa, Artificial Sequence
  US28-Nb3 VHH
EVQLVESGGGLVQAGGSLRLSCVVSGTIFSYTGVAWYRQTSGNQREWVASATNDGGTKFADSVKGRFTISRDNAKKTVHLQMNNLNAEDAAVYYCKTGRFWGRGTLVTVSS

SEQ ID NO: 21 - protein, 111 aa, Artificial Sequence
  US28-Nb4 VHH
EVQLVESGGGLVQAGGSLRLSCVVSGTIFSYTGVAWYRQPSGKQREWVASATNDGGTKFADSVKGRFTISRDNAKKTVYLQMNNLDADDTAVYYCKTGRFWGRGTLVTVSS

SEQ ID NO: 22 - protein, 114 aa, Artificial Sequence
  consensus VHH
  MISC_FEATURE (14)..(14): X can be any amino acid or absent
  MISC_FEATURE (21)..(21): X can be any amino acid or absent
  MISC_FEATURE (23)..(23): X can be any amino acid or absent
  MISC_FEATURE (27)..(29): X can be any amino acid or absent
  MISC_FEATURE (32)..(33): X can be any amino acid or absent
  MISC_FEATURE (41)..(41): X can be any amino acid or absent
  MISC_FEATURE (43)..(44): X can be any amino acid or absent
  MISC_FEATURE (46)..(47): X can be any amino acid or absent
  MISC_FEATURE (49)..(50): X can be any amino acid or absent
  MISC_FEATURE (53)..(54): X can be any amino acid or absent
  MISC_FEATURE (56)..(56): X can be any amino acid or absent
  MISC_FEATURE (59)..(59): X can be any amino acid or absent
  MISC_FEATURE (61)..(63): X can be any amino acid or absent
  MISC_FEATURE (72)..(72): X can be any amino acid or absent
  MISC_FEATURE (76)..(76): X can be any amino acid or absent
  MISC_FEATURE (78)..(78): X can be any amino acid or absent
  MISC_FEATURE (80)..(80): X can be any amino acid or absent
  MISC_FEATURE (84)..(84): X can be any amino acid or absent
  MISC_FEATURE (87)..(87): X can be any amino acid or absent
  MISC_FEATURE (89)..(90): X can be any amino acid or absent
  MISC_FEATURE (102)..(103): X can be any amino acid or absent
  MISC_FEATURE (106)..(106): X can be any amino acid or absent
  MISC_FEATURE (109)..(109): X can be any amino acid or absent
EVQLVESGGGLVQXGGSLRLXCXVSGXXXIFXXTGVAWYRXQXXGXXRXXVAXXTXDGXTXXXDSVKGRFTXSRDXAXKXVYLXMNXLXXEDTAVYYCKTGXXWGXGTXVTVSS

SEQ ID NO: 23 - protein, 32 aa, Artificial Sequence
  FR1`
  MISC_FEATURE (14)..(14): X is A or P
  MISC_FEATURE (21)..(21): X is S or A
  MISC_FEATURE (23)..(23): X is V or A
  MISC_FEATURE (27)..(27): X is T or P
  MISC_FEATURE (28)..(28): X is G or absent
  MISC_FEATURE (29)..(29): X is L or absent
  MISC_FEATURE (32)..(32): X is S or K
EVQLVESGGGLVQXGGSLRLXCXVSGXXXIFX

SEQ ID NO: 24 - protein, 15 aa, Artificial Sequence
  FR2
  MISC_FEATURE (4)..(4): X is R or absent
  MISC_FEATURE (6)..(6): X is T or V
  MISC_FEATURE (7)..(7): X is S or P
  MISC_FEATURE (9)..(9): X is K or A
  MISC_FEATURE (10)..(10): X is Q or K
  MISC_FEATURE (12)..(12): X is E or G
  MISC_FEATURE (13)..(13): X is W or L
WYRXQXXGXXRXXVA

SEQ ID NO: 25 - protein, 37 aa, Artificial Sequence
  FR3
  MISC_FEATURE (1)..(1): X is F or Y
  MISC_FEATURE (2)..(2): X is A or G
  MISC_FEATURE (11)..(11): X is I or V
  MISC_FEATURE (15)..(15): X is N or I
  MISC_FEATURE (17)..(17): X is K or A
  MISC_FEATURE (19)..(19): X is T or R
  MISC_FEATURE (23)..(23): X is Q or E
  MISC_FEATURE (26)..(26): X is N or D
  MISC_FEATURE (28)..(28): X is N or R
  MISC_FEATURE (29)..(29): X is A or S
XXDSVKGRFTXSRDXAXKXVYLXMNXLXXEDTAVYYC

SEQ ID NO: 26 - protein, 11 aa, Artificial Sequence
  FR4
  MISC_FEATURE (3)..(3): X is R or G
  MISC_FEATURE (6)..(6): X is L or Q
WGXGTXVTVSS

(19) United States
(12) Patent Application Publication
(10) Pub. No.: US 2022/0324947 A1
(43) Pub. Date: Oct. 13, 2022

(54) Identification and Elimination of HCMV-Infected Cells

(71) Applicants: Stichting VU, Amsterdam (NL); Cambridge Enterprise Limited, Cambridge (GB)

(72) Inventors: Martine Joyce Smit, Amsterdam (NL); Timo Werner Marcella De Groof, Gent (BE); Raimond Heukers, Odijk (NL); John Sinclair, Cambridge (GB); Elizabeth Elder, Stockholm (SE)

(21) Appl. No.: 17/632,023

(22) PCT Filed: Aug. 5, 2020
(86) PCT No.: PCT/NL2020/050497

§ 371 Date: Feb. 1, 2022

(30) Foreign Application Priority Data
Aug. 5, 2019 (EP) 19190047.1
May 7, 2020 (EP) 20173425.8

(51) Int. Cl. C07K 16/08; C12N 7/00; G01N 33/50

(52) U.S. Cl. CPC C07K 16/088; C12N 7/00; G01N 33/5091; C07K 2317/21; C07K 2317/24; C07K 2317/35; C12N 2710/16151

(57) ABSTRACT

The invention relates to the use of a single heavy chain variable domain antibody against human cytomegalovirus protein US28, which antibody binds to the extracellular region including, for example, the N-terminal extracellular region and/or the extracellular loops of US28, for isolation of cells that are infected with cytomegalovirus and/or for ex vivo reactivation of cytomegalovirus in latently infected cells. The invention further relates to the anti-US28 antibody for use in a method of reactivating cytomegalovirus in infected cells, or in a method of eliminating infected cells. The invention further relates to a tissue, organ, or cells such as bone marrow stem cells, from which cells that were infected with CMV have been removed with the use of the anti-US28 antibody.

### FIELD

The invention relates to the field of virology. More specifically, the invention relates to a single heavy chain variable domain antibody which binds to the extracellular side, including for example the N-terminus and/or the extracellular loops, of G protein-coupled receptor US28 that is encoded by human cytomegalovirus (HCMV). These antibodies are useful in recognition and isolation of cells that are infected with HCMV and/or for reactivation of HCMV in infected cells and/or elimination of infected cells.

### 1 INTRODUCTION

The human cytomegalovirus (HCMV; HHV-5) is an important herpesvirus with a prevalence range of 50 to 90% of the worldwide population (Sinclair and Sissons, 2006. J Gen Virol 87: 1763-791). In healthy individuals, HCMV establishes a persistent, lifelong and asymptomatic infection (Sinclair and Sissons, 2006. Ibid; Gandhi and Khanna, 2004. Lancet Infect Dis 4: 725-382). During primary HCMV infection, multiple cell types, including fibroblasts, epithelial cells, smooth muscle cells and endothelial cells, can be infected by means of viral entry mediated by glycoprotein complexes on the viral envelope (Krishna et al., 2018. Viruses 10: 445; Vanarsdall and Johnson, 2012. Curr Opin Virol 2: 37-42). In healthy individuals, this primary infection is usually sub-clinical, due to a robust innate and adaptive host immune response. However, not all infected cells will be cleared and the virus will persist as a latent infection in a small percentage of CD34+ progenitor cells and the derivative CD14+ monocytes (Taylor-Wiedeman et al., 1991. J Gen Virol 72: 2059-64; Hahn et al., 1998. Proc Natl Acad Sci USA 95: 3937-42) resulting in the carriage of the viral genome but no production of viral particles. Reactivation of the virus towards a lytic infection can occur upon differentiation of the CD14+ monocytes into macrophages or dendritic cells but this will subsequently lead to the clearance of these cells by the immune system (Poole et al., 2015. J Infect Dis 211: 1936-42; Reeves and Sinclair, 2013. J Virol 87: 10660-7). One key feature during latency is the suppression of the immediate early (IE) gene expression, which indicates the initiation of reactivation, and is controlled by the major immediate early promoter (MIEP) (Reeves et al., 2005. Proc Natl Acad Sci USA 102: 4140-5; Reeves and Sinclair, 2010. J Gen Virol 91: 599-604; Goodrum et al., 2002. Proc Natl Acad Sci USA 99: 16255-60; see also FIG. 1). Suppression or initiation of IE gene expression is controlled by modifications of the chromatin structure around the MIEP including histone methylation, acetylation and phosphorylation which will result in a closed or open chromatin structure (Reeves et al., 2005. Ibid; Rossetto et al., 2013. PLoS Pathog 9: e1003366; Rauwel et al., 2015. Elife 4: :e06068; Kew et al., 2014. PLoS Pathog 10: e1004195; Krishna et al., 2016. Sci Rep 6: 24674). Although primary infection and reactivation generally do not cause any harm in healthy individuals, this is not the case for immunocompromised (HIV/AIDS patients), immunosuppressed persons such as transplant recipients, or immune-naïve persons (fetus/neonates) (Sinclair and Sissons, 2006. Ibid). Especially in (solid) organ transplant patients, reactivation can pose a great risk leading to the manifestation of diverse clinical consequences including pneumonitis, hepatitis, nephritis but also allograft rejection (Gandhi and Khanna, 2004. Ibid; Griffiths et al., 2015. J Pathol 235: 288-97; Ramanan and Razonable, 2013. Infect Chemother 45: 260-71). These HCMV-associated diseases are currently being treated with antivirals such as ganciclovir, foscarnet, and letermovir, which target reactivated, lytic virus (Marty et al., 2017. N Engl J Med 377: 2433-2444). However, there no current antiviral treatments which target the latent HCMV reservoir to reduce the HCMV-mediated clinical manifestations during organ transplantation.

There is thus a need for tools that allow the isolation and analysis of latently infected cells, and of methods and means to target latently infected cells to reduce the HCMV-mediated clinical manifestations before or during organ transplantation.

### 2 BRIEF DESCRIPTION OF THE INVENTION

The invention is directed to the use of a single heavy chain variable domain antibody against human cytomegalovirus protein US28, which binds to the extracellular region including, for example, the N-terminal extracellular region and/or the extracellular loops of US28, for isolation of cells that are infected with cytomegalovirus and/or for ex vivo reactivation of cytomegalovirus in infected cells. Such single heavy chain variable domain antibody preferably is coupled to a tag to allow identification and isolation of HCMV-infected cells, be they either latently infected cells, or cells in which the virus is at least partially reactivated. The invention further provides a single heavy chain variable domain antibody against human cytomegalovirus protein US28, which antibody binds to the extracellular region including, for example, the N-terminal extracellular region and/or the extracellular loops of US28, for use in a method for in vivo reactivation of cytomegalovirus in infected cells, or in a method of eliminating infected cells, be they either latently infected cells, or cells in which the virus is at least partially reactivated.

Said single heavy chain variable domain antibody preferably comprises complementarity-determining regions (CDRs) having amino acid sequences F/YTGVA for CDR1; L/T/SI/T/ATG/NDGA/GTR/K for CDR2; and KTGE/RY/F for CDR3.

Said single heavy chain variable domain antibody preferably comprises human or humanized frame work regions.

Said single heavy chain variable domain antibody preferably is fused to an immunoglobulin Fc region or functional part thereof, preferably wherein the Fc region or functional part thereof is from, or derived from, IgG1, IgG2, IgG3, IgG4. Said Fc region preferably is a human or humanized Fc region, or functional part thereof.

Said single heavy chain variable domain antibody preferably is part of a bi- or multivalent antibody. It was found that a single heavy chain variable domain antibody against human cytomegalovirus protein US28, which antibody binds to the extracellular region including, for example, the N-terminal extracellular region and/or the extracellular loops of US28, acts as an antagonist of US28 as a monovalent antibody, but as an inverse agonist of US28 when provided as a bivalent antibody.

When applied as inverse agonist, the inactivation of US28 in latently infected cells is not complete, resulting in only partial reactivation of cytomegalovirus in these infected cells. The incomplete reactivation induces immediate early (IE) gene expression which is sufficient for the individual's own immune system to kill the cells in which cytomegalovirus is partially reactivated.

In an embodiment, the antibody for use according to the invention is for detection, enrichment and/or purification of infected cells, or to eliminate infected cells that are present in an individual by US28-targeted leukapheresis. Said cells are either latently infected with cytomegalovirus, or cells in which the virus is at least partially reactivated.

In an embodiment, the antibody for use according to the invention is for reactivating cytomegalovirus in latently infected cells, or for eliminating latently infected cells and/or reactivated cells that are present in an individual.

In an embodiment, the antibody for use according to the invention is for reactivating cytomegalovirus in latently infected cells, or for eliminating latently infected cells and/or reactivated cells that are present in an organ that is to be transplanted.

In an embodiment, the antibody for use according to the invention is for reactivating cytomegalovirus in latently infected cells, or for eliminating latently infected cells and/or reactivated cells that are present in stem cells, preferably bone marrow stem cells, that are to be transplanted.

An antibody for use according to the invention may be administered in combination with an anti-viral agent and/or a histone deacetylase inhibitor.

An antibody for use according to the invention may be coupled to a cytotoxic drug as effector molecule. Such effector molecules include both toxic proteins as well as toxic chemicals, including those commonly used in conventional antibody-drug conjugates (ADC). Said effector molecule preferably is a photosensitizer, preferably a photosensitiser that can be activated by exposure to light for use in photodynamic therapy (PDT).

The invention further provides a tissue, organ, or cells such as bone marrow stem cells, from which individual cells that were infected with CMV have been removed with the use of a single heavy chain variable domain antibody against human cytomegalovirus protein US28.

### 3 FIGURE LEGENDS

FIG. 1. Overview of US28 signaling pathways in latent and cancer setting. US28 suppresses the major immediate early promotor (MIEP) and subsequent immediate early (IE) gene expression in early myeloid cells. US28 attenuates several pathways including the Extracellular Signal-Regulated Kinases (ERK)1/2, c-fos and Nuclear Factor-κB (NF-κB) pathway. US28 inhibits phosphorylation (P) of ERK1/2 which inhibits phosphorylation of Mitogen and Stress Activated Protein Kinase (MSK) and subsequent cAMP Response Element Binding Protein (CREB) phosphorylation. US28 activation also leads to reduced c-fos expression. Reduced c-fos levels leads to less dimer formation of c-fos and c-jun. In addition, US28 signaling inhibits entering of NF-κB in the nucleus. By activation of the Signal Transduced and Activator of Transcription 3 (STATS)-inducible Nitric Oxide Synthase (iNOS) pathway, US28 activation leads to increased nitric oxide (NO) levels resulting in suppression of the MIEP. By suppression of MIEP, no IE expression is observed resulting in a latent infection in early myeloid cells.

FIG. 2. VUN100 and VUN100b bind specifically to US28 in THP-1 cells. Immunofluorescence microscopy binding of an irrelevant nanobody (Irr Nb), VUN100 or bivalent VUN100 (VUN100b) to THP-1 mock transduced cells (A) and THP-1 US28 transduced cells (B). Nanobody (Nb) binding was detected using the Myc-tag and an anti-Myc antibody. US28 expression was detected using an anti-US28 antibody.

FIG. 3. VUN100b transiently reactivates immediate early expression. A) CD14+ monocytes were isolated, infected with HCMV IE-YFP and treated with an irrelevant nanobody, VUN100 or VUN100b. Two days post infection, cells were fixed and stained for immediate early (IE) expression. B) CD14+ monocytes were isolated, infected with HCMV IE-YFP and treated with PMA, an irrelevant nanobody, VUN100 or VUN100b. IE-positive cells were counted 6 days post infection.

FIG. 4. VUN100b induces immediate early and late gene expression but no true late gene expression. CD14+ monocytes were isolated, infected with HCMV IE-YFP and treated with phorbol myristate acetate (PMA), an irrelevant nanobody (Nb), VUN100 or VUN100b. RNA was isolated and analyzed by RT-qPCR 6 days post infection. Immediate early UL123 gene expression (A), latent UL138 gene expression (B), late UL44 gene expression (C) and true late UL99 gene expression (D) was assessed. Values were normalized to GAPDH gene expression.

FIG. 5. HCMV-infected CD14+ monocytes are targets for HCMV specific T-cells upon US28 nanobody treatment. A) CD14+ monocytes were isolated, infected with HCMV IE2-YFP and treated with nanobodies for 6 days post infection. After nanobody (Nb) treatment, cells were co-cultured with CD4/CD8+ T cells or T-cell depleted peripheral blood mononuclear cells (PBMCs). After 2 days, T cells or PBMCs were removed and CD14+ monocytes were differentiated into immature dendritic cells by addition of interleukin-4 (IL-4) and granulocyte-macrophage colony-stimulating factor (GM-CSF). Lipopolysacharide (LPS) was added to induce mature dendritic cells and induce immediate early (IE) expression and IE-positive cells were counted. B) Counting of IE-positive CD14+ monocytes before co-culture with CD4/CD8+ T cells or T-cell depleted PBMCs. C) Counting of IE-positive nuclei after 9 days of co-culture of T-cells/PBMCs, differentiation and addition of LPS.

FIG. 6. T cell co-culture experiment of CD14+ monocytes infected with HCMV UL32-GFP virus. A) Counting of UL32-GFP positive CD14+ monocytes before co-culture with CD4/CD8+ T cells. CD14+ monocytes were treated with an irrelevant nanobody (Irr Nb) or VUN100b for 6 days post infection. B) Quantification of HCMV genomes after co-culture of CD4/CD8+ T cells, differentiation of CD14+ monocytes to mature dendritic cells and co-culturing of mature dendritic cells with Hff1 cells. Data is plotted as mean±S.D. Statistical analyses were performed using unpaired two-tailed t-test. ns, p>0.05; **, p<0.01.

FIG. 7. VUN100b induces immediate early expression but no full viral reactivation after establishment of latency. A) CD14+ monocytes were isolated, infected with HCMV IE2-YFP. Six days post infection, cells were treated with an irrelevant nanobody (Irr Nb), VUN100, VUN100b or 20 ng/ml PMA (PMA). IE-positive nuclei were counted 8 days post infection. B) Eight days post infection, nanobody-treated or PMA-treated monocytes were co-cultured with Hff1 fibroblasts. IE-focus formation was quantified after 8 days of co-culturing. Data is plotted as mean+SD. Statistical analyses were performed using unpaired two-tailed t-test. ns, p>0.05; *, p<0.05; **, p<0.01; ****, p<0.001.

FIG. 8. Immediate-early positive CD14+ monocytes 6 days post infection. CD14+ monocytes were isolated and seeded. The next day, cells were uninfected (Uninf) or infected with HCMV IE2-YFP for 2 hours. IE2-positive nuclei were counted 6 days post infection. Data is plotted as mean+SD.

FIG. 9. Immediate-early positive CD14+ monocytes 6 days post infection. CD14+ monocytes were isolated and infected with HCMV Titan wildtype (WT) or Titan AUS28 virus for 2 hours the next day. Six days post infection, cells were untreated (Untr), treated with the irrelevant nanobody (Irr Nb), VUN100, VUN100b or PMA. IE-positive nuclei were counted 3 days post infection. Data is plotted as mean±S.D. Statistical analyses were performed using unpaired two-way ANOVA with Tukey's multiple comparison test. ns, p>0.05; ***, p<0.001; ****, p<0.0001.

### 4 DETAILED DESCRIPTION OF THE INVENTION

### 4.1 Definition

The term “antibody” as used herein, refers to an antigen binding protein comprising at least a heavy chain variable region (Vh) that binds to a target epitope. The term antibody includes monoclonal antibodies comprising immunoglobulin heavy and light chain molecules, single heavy chain variable domain antibodies, and variants and derivatives thereof, including chimeric variants of single heavy chain variable domain antibodies and multivalent and/or multispecific variants of single heavy chain variable domain antibodies.

The term “single heavy chain variable domain antibody”, as is used herein, refers to a heavy chain only antibody that is devoid of light chains. Preferably said single heavy chain variable domain antibody is an antibody of the type that can be found in Camelidae or cartilaginous fish which are naturally devoid of light chains, or a synthetic antibody which can be constructed accordingly. An alternative term for a single heavy chain variable domain antibody is Variable Heavy chain Homodimer or VHH.

As described herein, the amino acid sequence and structure of a heavy chain variable domain, including a VHH, can be considered—without however being limited thereto—to be comprised of four framework regions or ‘FR’, which are referred to in the art and herein as ‘Framework region 1’ or ‘FR1’; as ‘Framework region 2’ or'FR2′; as ‘Framework region 3’ or ‘FR3’; and as ‘Framework region 4’ or ‘FR4’, respectively; which framework regions are interrupted by three complementary determining regions or ‘CDR’ s′, which are referred to in the art as ‘Complementarity Determining Region 1’ or ‘CDR1’; as ‘Complementarity Determining Region 2’ or ‘CDR2’; and as ‘Complementarity Determining Region 3’ or ‘CDR3’, respectively. For the purpose of this patent application, amino acid residues 31-35 of VHH are defined as CDR1, amino acid residues 50-57 of VHH are defined as CDR2, and amino acid residues 97-100 of VHH are defined as CDR3, with the amino acid residue numbering according to according to Riechmann and Muyldermans, 1999 (Riechmann and Muyldermans, 1999. J Immunol Methods 23: 25-38), as is indicated in Table 1.

The total number of amino acid residues of a VHH is typically in the region of 110-120, is preferably 111-115, and is most preferably 113.

The term ‘binding’ as used herein in the context of binding between an antibody, preferably a VHH, and an epitope of US28 as a target, refers to the process of a non-covalent interaction between molecules. Preferably, said binding is specific. The terms ‘specific’ or ‘specificity’ or grammatical variations thereof refer to the number of different types of antigens or their epitopes to which a particular antibody such as a VHH can bind. The specificity of an antibody can be determined based on affinity. A specific antibody preferably has a binding affinity Kd for its specific epitope of less than 10⁻⁷ M, preferably less than 10⁻⁸ M.

The term affinity refers to the strength of a binding reaction between a binding domain of an antibody and an epitope. It is the sum of the attractive and repulsive forces operating between the binding domain and the epitope. The term affinity, as used herein, refers to the apparent binding affinity, which is determined as the equilibrium dissociation constant (Kd).

The term epitope or antigenic determinant refers to a part of an antigen that is recognized by an antibody. The term epitope includes linear epitopes and conformational epitopes, also referred to as continuous and discontinuous epitopes respectively. A conformational epitope is based on 3-D surface features and shape and/or tertiary structure of the antigen. A posttranslational modification, such as phosphorylation, glycosylation, methylation, acetylation and lipidation, may be relevant for an epitope for recognition by a specific antibody.

The term CMV refers to a virus of the genus Cytomegalovirus, which currently harbours eight species. Human cytomegalovirus (HCMV), also termed human herpesvirus 5 (HHV-5) is the type species.

The term US28 refers to a G protein-coupled receptor that is encoded by herpesviruses, especially CMV. US28 is a rare multi-chemokine family binding receptor with the ability to bind ligands such as CCL2/MCP-1, CCL5/RANTES, and CX3CL1/fraktalkine as ligands. Ligand binding to US28 activates cell-type and ligand-specific signalling pathways leading to cellular proliferation and migration, which is an important example of receptor functional selectivity. Additionally, US28 has been demonstrated to constitutively activate Gαq, phospholipase C

(PLC) and NF-κB signaling pathways, amongst others.

The term “extracellular loops”, as is used herein, refers to the parts of the protein that are located on the outer side of a plasma membrane. Being a G protein-coupled receptor, US28 comprises 7 transmembrane domains and the extracellular loops comprise the N-terminal part in front of the first transmembrane domain, the part between the second and third transmembrane domains, the part between the fourth and fifth transmembrane domains, and the part between the sixth and seventh transmembrane domains.

The term “transmembrane domain”, as is used herein, refers to a membrane-spanning protein domain. A transmembrane domain in general comprises non-polar and/or hydrophobic amino acid residues. The presence of a transmembrane domain can be predicted using, for example, hydrophobicity analysis. Computational resources are available, such as HMMTOP, TMpred, TMHMM and TopPred2, that may help in predicting transmembrane domains in protein sequences. The transmembrane domains of US28 are known and have been described in, for example, Rosenkilde et al., 2008. Brit J Pharm 153: S154—S166. Burg et al., 2015. Science 347: 1113-7.

The term “N-terminal extracellular region of US28” refers to the N-terminal region on US28 with the amino acid sequence N-terminus MTPTTTTA/TELTTEFD/EYDD/E/LE/D/GATP, in which NT, D/E, D/E/L and E/D/G indicate alternative amino acid residues at a position, as present in different HCMV strains, indicated by the standard one letter amino acid code.

The term “inverse agonist”, as is used herein, indicates that binding of an antibody according to the invention to the receptor not only blocks the effects of agonist-binding, such as binding of CCL2/5 and/or CX3CL1, but also inhibits basal activity of the receptor through downstream pathways such as NF-κB.

The term “leukapheresis”, as is used herein, refers to the separation and isolation of white blood cells are separated from blood. It is a specific type of apheresis, which generally describes the separation of a particular constituent from blood and the return if the remainder to the circulation.

### 4.2 Anti-US28 Antibodies

Described herein is a specific class of antibodies, namely single heavy chain variable domain antibody antibodies or VHH. The heavy chain variable domain antibodies were isolated from llamas and alpacas that were immunized with DNA constructs expressing US28 from HCMV. The camelids received a primary injection and at least one boost injection at 14 days after the primary injection, preferably 2-10 boost injections such as, for example, five boost injections.

Immune phage display libraries were generated from these animals 14 days after the last boost injection. These libraries were screened for binding to US28, and for their ability to displace a radiolabelled chemokine CCL5 (¹²⁵I-CCL5) from US28-expressing cells. This resulted in the isolation of one VHH that fully inhibited the binding of radiolabelled CCL5 or CX3CL1 to US28.

Described herein is a single heavy chain variable domain antibody against human cytomegalovirus protein US28, which antibody binds to extracellular loops including, for example, an N-terminal extracellular region and/or the extracellular loops, of US28.

Said anti-US28 heavy chain variable domain antibody or VHH preferably has a binding affinity of at most 10⁻⁶ M, more preferred at most 10⁻⁷M, more preferred at most 10⁻⁸ M, more preferred at most 10⁻⁹ M, more preferred at most 10⁻¹⁰ M.

A VHH according to the invention preferably comprises CDR1, CDR2 and CDR3 amino acid sequences XTGVA, preferably F/YTGVA for CDR1; XXTXDGXTX, preferably L/T/SI/T/ATG/NDGA/GTR/K for CDR2; and KTGXX, preferably KTGE/RY/F for CDR3, or a derivative thereof. Said CDR sequences preferably are present in consensus framework regions (FR) as presented in Table 1. Further preferred CDR sequences, and full length sequence of the variable VHH regions, are provided in Table 1. A most preferred VHH according to the invention comprises amino acid sequences FTGVA for CDR1, LITGDGATR for CDR2, and KTGEY for CDR3. A most preferred VHH according to the invention comprises amino acid sequences of VUN100 as indicated in Table 1.

A remarkable finding was that the identified VHHs, which originated from different lamas and were derived from different V-genes, are characterized by similar CDR1 and CDR3 sequences, and related CDR2 sequences. In addition, VHHs were found to act as antagonists of US28 as monovalent antibodies, and as inverse agonists of US28 as bivalent antibodies, which reduces constitutive US28 signaling. Hence, a preferred single heavy chain variable domain antibody binds to extracellular loops including, for example, an N-terminal extracellular region, of US28 and acts as an antagonistic or inverse agonistic antibody.

A preferred derivative may comprise alterations of the amino acid sequence of the CDRs to increase their efficiency, affinity and/or physical stability including, for example a conservative derivative. The term “conservative derivative”, as used herein, denotes the replacement of an amino acid residue by another, biologically similar residue. Examples of conservative derivatives include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like. The CDR sequences of a preferred derivate, preferably a conservative derivative, preferably are more than 80% identical, more preferably are more than 90% identical, more preferably are more than 95% identical to the amino acid sequences of CDR1, CDR2 and CDR3 indicated herein above.

In addition, said antagonistic, preferably inverse agonistic, anti-US28 antibody may comprise a tag at its N-terminus and/or its C-terminus. Said tag may be added to the protein by genetic engineering to allow the antibody to attach to a column specific to the tag and therefore be isolated from impurities. In addition, the tag may be used to detect a tagged antibody, for example in Western blotting experiments or in immunohistochemistry.

Conventional tags for proteins, such as histidine tag, can be used with an affinity column that specifically captures the tagged protein. The tagged protein is subsequently eluted from said column, eg., a Ni-IDA column for a histidine tag, using a decoupling reagent according to the specific tag (eg., immidazole for histidine tag). Suitable tags include one or more of a his-tag (HHHHHH), c-myc domain (EQKLISEEDL), hemagglutinin tag (YPYDVPDYA), maltose-binding protein, glutathione-S-transferase, maltose-binding protein, FLAG tag, biotin acceptor peptide, streptavidin-binding peptide and calmodulin-binding peptide, as presented in Chatterjee, 2006. Cur Opin Biotech 17, 353-358). Methods for employing these tags are known in the art and may be used for purifying and/or detection of said VHH antibody.

Said antibody may also comprise an immunoglobulin Fc region for eliminating cells carrying US28 proteins on their surface via antibody-dependent cell-mediated cytotoxicity (ADCC) routes and/or complement dependent cytotoxicity (CDC) routes.

In a preferred embodiment, said antibody comprises an immunoglobulin Fc region or functional part thereof of an immunoglobulin heavy chain. The Fc region or functional part thereof is preferably derived from IgG1, IgG2, IgG3, IgG4, IgM, IgD, IgA or IgE. It is further preferred that the Fc region or part thereof is a human Fc region or part thereof or a camelid Fc region or part thereof, for example a lama Fc region or part thereof. Said camelid Fc region or part thereof preferably is humanized.

A single heavy chain variable domain is preferably connected to a Fc region or functional part thereof via a hinge region. A preferred hinge region is the hinge region of a camelid or human immunoglobulin heavy chain molecules from IgG1, IgG2, IgG3, IgG4, IgM, IgD, IgA or IgE, most preferred from IgG1. A hinge region of a camelid immunoglobulin heavy chain molecule preferably is humanized.

A preferred part of an Fc region is the region comprising the CH2 domain, the CH3 domain, or the CH2 and CH3 domains of IgGs, preferably IgG1 or IgG3, most preferably CH2 and CH3 domains of human IgG1.

A further preferred antibody is a bi- or multivalent antibody comprising an anti-US28 single heavy chain variable domain as described. Said bi- or multivalent antibody preferably is a bispecific or multispecific antibody comprising two or more single heavy chain variable domains. Said single heavy chain variable domains may be the same, or different recognizing the same or different epitopes on a US28 molecule, preferably on the extracellular region of US28. A bi- or multivalent antibody preferably comprises two or more single heavy chain variable domains as described. However, a VHH of the invention may also be combined with other, preferably non-competing and non-interfering anti-US28 VHH, or VHH targeting other proteins such as VHH specifically binding soluble proteins expressed in blood, like for example serum albumin, or proteins expressed on the surface of cells, like Fc-binding proteins on immune cells or other receptor proteins embedded in the cell membrane.

Said two or more single heavy chain variable domains are preferably fused to the Fc region or part thereof, preferably comprising the C2 and C3 domains of IgGs, preferably IgG1 or IgG3, most preferably human C2 and C3 domains. The constant region that is fused to the single heavy chain variable domains preferably comprises a dimerization or multimerization motif. Alternatively, a bi- or multivalent antibody may be generated by chemical cross-linking or by a heterologous dimerization or multimerization domain comprising, for example, a leucine zipper or jun-fos interaction domain (Pack and Plückthun, 1992. Biochemistry 31, 1579-1584; de Kruif and Logtenberg, 1996. JBC 271: 7630-7634). A further preferred bi- or multivalent antibody is a bihead or a multihead VHH, for example a trihead VHH, as described in WO2000/024884.

The bihead or multihead antibodies preferably comprise a linking group which provides conformational flexibility so that each of the single heavy chain variable domains can interact with its epitope. A preferred linker group is a linker polypeptide comprising from 1 to about 60 amino acid residues, preferably from 10 to about 40 amino acid residues, most preferred about 35 amino acid residues such as 30 amino acid residues, 31 amino acid residues, 32 amino acid residues, 33 amino acid residues, 34 amino acid residues, 35 amino acid residues, 36 amino acid residues, 37 amino acid residues, 38 amino acid residues, or 39 amino acid residues. Some preferred examples of such amino acid sequences include Gly-Ser linkers, for example of the type (Glyx Sery)z such as, for example, (Gly4 Ser)3, (Gly4 Ser)7 or (Gly3 Ser2)3, as described in WO 99/42077, and the GS30, GS15, GS9 and GS7 linkers described in, for example, WO 06/040153 and WO 06/122825, as well as hinge-like regions, such as the hinge regions of naturally occurring heavy chain antibodies or similar sequences as described in WO 94/04678.

### 4.3 Use of Anti-US28 Antibodies

HCMV is a betaherpesvirus (Krishna et al., 2017. MBio 8: 1-21). A betaherpesvirus is characterized by being slowly progressive, having long reproductive life cycle and being able to undergo latency (Whitley, Herpesviruses. In: Medical Microbiology. 4th edition, Baron S1, editor. Galveston (Tex.): University of Texas Medical Branch at Galveston; 1996). This leads to three possible phenomena in the life cycle of HCMV, namely primary infection, latency and reactivation. HCMV is widely spread with 50-90% of the world population being HCMV positive (Krishna et al., 2017. MBio 8: 1-21). However, the infection hardly causes any clinical syndromes due to an efficient immune system response (Krishna et al., 2017. Nat Commun 8: 1-9). The innate and adaptive immune response leads to T cell recruitment and activation of natural killer (NK) cells (Elder et al., 2019. iScience 12: 13-26). These T cells will recognize HCMV antigens leading to clearance of most of the infected cells and virus particles (Slobedman and Mocarski, 1999. J Virol 73: 4806-4812). However, not all HCMV infected cells are cleared by the immune system (Krishna et al., 2017. Nat Commun 8: 1-9). Latently-infected CD34+ progenitor cells and their derivative, CD14+ monocytes, evade the immune system leading to a lifelong viral persistence (Krishna et al., 2017. MBio 8: 1-21; Krishna et al., 2017. Nat Commun 8: 1-9).

Latently infected cells express US28 intracellularly and on their surface. An anti-US28 antibody according to the invention which binds to the extracellular domain of US28, may thus be used to isolate cells that express US28 on their surface, such as cells that are latently infected with HCMV, or cells in which the virus is reactivated. Said cells preferably are infected, primary cells, preferably latently infected primary cells or primary cells in which the virus is reactivated.

Said latently infected cells, including monocytes, may be isolated directly from a bodily fluid such as blood, urine, milk, cerebrospinal fluid, interstitial fluid, lymph, amniotic fluid, bile, cerumen, feces, female ejaculate, gastric juice, mucus pericardial fluid, pleural fluid, saliva, semen, smegma, sputum, synovial fluid, sweat, tears, and/or vaginal secretion.

As an alternative, or in addition, said latently infected cells and/or (partially) reactivated cells may be isolated from a tissue or organ including, for example, bone marrow including hematopoietic stem cells, kidney, liver, stratum corneum, heart, heart valve, skin, lung, pancreas, small intestine, bone, tendon, cartilage, veins and arteries.

To provide individual cells from a tissue or organ, said tissue or organ may be minced prior to isolation of the latently infected cells, to obtain smaller fragments of the tissue, preferably approximately 0.5 to 2 mm in diameter. Mincing may be performed by any suitable method, for instance using scissors, razor blades, a scalpel, straining through a steel or nylon mesh screen or sieve, or disaggregating it through a needle.

The tissue, organ, or fragments thereof, may be subjected to a treatment to increase extracellular matrix permeability prior to subjecting it to a digestion enzyme. Said treatment may include contacting the tissue or organ with an acid, a base, dimethyl sulfoxide (DMSO), cathepsin, glycerol, or cations such as include Na⁺, K⁺, NH⁴⁺, Pb²⁺, Mg²⁺, Zn²⁺, Fe²⁺, Cd²⁺, and Cu²⁺ ions, or any other agent which may increase the Donan osmotic pressure of the extracellular matrix or cause the extracellular matrix to swell, prior to subjecting it to a digestion enzyme, as is known to a person skilled in the art. After the permeability of the tissue is increased, the tissue, organ or parts thereof may be washed with for instance phosphate buffered saline before subjecting it to a digestion enzyme.

The tissue, organ or parts thereof may then be incubated in a digestion solution, preferably for a period of less than two hours, preferably for a period of from 10 to 30 minutes. The digestion solution comprises one or more enzymes chosen from the group consisting of collagenases, pronases, dispases, trypsins, hyaluronidases, chondroitinases, elastases, and heparitinases. The type of enzyme will depend on the type of tissue used. It is also contemplated to use different enzymes sequentially or simultaneously. The conditions (e.g. pH and temperature) under which single cells are generated from an organ or tissue will be chosen such that they are optimal for the cells that are being isolated and for the digestion enzyme and possible other agents used. To this end, the digestion solution may further comprise buffering agents which help to maintain the pH in the range which approximates physiological conditions. They are preferably present at concentration ranging from about 1 mM to about 100 mM. Suitable buffering agents for use in the present invention include both organic and inorganic acids and salts thereof such as citrate, succinate, tartrate, fumarate, gluconate, oxalate, lactate, acetate phosphate and borate buffers. Additionally, there may be mentioned, histidine, glycine and urea buffers and buffers such as Tris, MOPS and HEPES.

The digestion solution may further comprise such compounds as: chelating agents, e.g. diethylenetriaminepentacetic acid (DTPA), ethylenediaminetetraacetic acid (EDTA), ethylene bis(oxyethylenenitrolo)tetraacetic acid (EGTA); reducing agents such as dithiotreitol, dithioerythritol, β-mercaptoethanol, glutathione, thioredoxine, cysteine, etc.; ions necessary for activation of the enzyme such as CaCl2, MgCl2, NaCl and/or KCl; and/or organic solvents or lipid/membrane modifying agents such as dimethyl sulfoxide (DMSO); nonionic detergents such as triton X-100; and/or osmoprotectants such as sucrose.

The resulting individual cells may then be harvested in the usual manner, e.g. by washing, filtration, and/or centrifuging. During this step, the cells are separated from the digestion enzyme and possible other agents used, thereby effectively ending the digestion process. If desired, the digestion enzyme may be inactivated prior to this separation step, e.g. by adjustment of the pH.

Individual cells from a bodily fluid, or from tissue or organ, may be isolated before or after a harvesting step by incubating the cells with an anti-US28 antibody that binds to the extracellular region including, for example, the N-terminal extracellular region of US28. The anti-US28 antibody may be labelled, for example with a fluorescent label, allowing the isolation of US28-expressing cells by FACS sorting. As an alternative, isolation using magnetic beads may be carried out by adding magnetic beads coated with said anti-US28 antibody to the cell suspension. The cells will bind to the magnetic beads via their membrane expressed extracellular region of US28. With the aid of a magnet, the beads with the cells attached thereto are concentrated, for example at the bottom of a tube, and the supernatant may be aspirated. The magnetic beads with the cells may be washed with e.g. PBS. This process can be repeated several times. Finally, the cells may be separated from the magnetic beads by trypsin treatment, competition by washing with epitope containing buffer (e.g. extracellular region of US28), washing with 1mM to 10 mM aqueous HC1 solution or, for example, with a mild and acidic glycine buffer (pH 2.5) that disrupts the binding of the antibody to US28.

### 4.4 Therapeutic Targeting of US28

Due to the important role of US28 in the HCMV lifecycle, multiple US28 inhibitors have been developed. In the past, multiple small molecules that blocked ligand binding to US28 and that at least partially inhibited the constitutive activity of US28 have been described (Hulshof et al., 2006. Bioorg Med Chem 14: 7213-30; Vischer et al., 2010. Bioorg Med Chem 18: 675-88). One of these compounds is the inverse agonist VUF2274, which completely inhibited US28 signaling (Casarosa et al., 2003. J Biol Chem 278: 5172-8). Interestingly, blocking US28 signaling by VUF2274 also resulted in full reactivation of latently HCMV-infected monocytes (Krishna et al., 2017. Ibid). Other studies have also reported the development of other inverse agonists based on flavonoids, biphenyl amides or CX3CR1 antagonists (Kralj et al., 2014. ChemMedChem 9: 151-68; Kralj et al., 2013. J Med Chem 56: 5019-32; Kralj et al., 2011. Bioorg Med Chem Lett 21: 5446-50). However, all these compounds had affinities and potencies in the micromolar range. In addition, they induced cytotoxic effects and/or aspecific binding to other

GPCRs, which makes them not well suited for in vivo use.

It will be clear to a person skilled in the art that latently infected cells and/or reactivated cells, be they partially or fully reactivated, may be isolated from, an/or removed from, for example, blood or hematopoietic stem cells, prior to transfusion or transplantation of said cells. Infected cells may be isolated and removed from blood or hematopoietic stem cells, for example by apheresis or leukapheresis. For example, said apheresis or leukapheresis device may comprise beads coated with an anti-US28 antibody that binds to the extracellular region US28. US28-expressing cells are absorbed onto the beads and removed by filtration or centrifugation.

TABLE 1
Amino acid sequences of isolated VHHs (Kabat numbering as applied for VHHs, according to Riechmann
and Muyldermans, 1999 (Riechmann and Muyldermans, 1999. J Immunol Methods 23: 25-38).

| | |
|---|---|
| | 1 10 20 ** 30 35 * 40 50 |
| VUN100 | EVQLVESGGGLVQPGGSLRLACAVSGPGLIFK FTGVA WYRRQVPGAKRGLVA LITGDGATR |
| US28-Nb1 | EVQLVESGGGLVQAGGSLRLSCVVSGT--IFS YTGVA WYR-QTSGKQREWVA TTTNDGGTK |
| US28-Nb2 | EVQLVESGGGLVQAGGSLRLSCVVSGT--IFS YTGVA WYR-QTSGNQREWVA TATNDGGTK |
| US28-Nb3 | EVQLVESGGGLVQAGGSLRLSCVVSGT--IFS YTGVA WYR-QTSGNQREWVA SATNDGGTK |
| US28-Nb4 | EVQLVESGGGLVQAGGSLRLSCVVSGT--IFS YTGVA WYR-QPSGKQREWVA SATNDGGTK |

| | 60 70 80 90 96 103 113 |
|---|---|
| VUN100 | YGDSVKGRFTVSRDIAAKRVYLEMNDLRSEDTAVYYC KTGEY WGQGTQVTVSS |
| US28-Nb1 | FADSVKGRFTISRDNAKKTVYLQMNNLNAEDTAVYYC KTGRF WGRGTLVTVSS |
| US28-Nb2 | FADSMKGRFTISRDNAKKTVYLQMNNLNAEDAAVYYC KTGRF WGRGTLVTVSS |
| US28-Nb3 | FADSVKGRFTISRDNAKKTVHLQMNNLNAEDAAVYYC KTGRF WGRGTLVTVSS |
| US28-Nb4 | FADSVKGRFTISRDNAKKTVYLQMNNLDADDTAVYYC KTGRF WGRGTLVTVSS |

Consensus

EVQLVESGGGLVQXGGSLRLXCXVSGXXXIFX XTGVA WYRXQXXGXXRXXVA XXTXDGXTX XXDSVKGRFTXSRDXAXKXVYLXMNXLXX

EDTAVYYC KTGXX WGXGTXVTVSS
Consensus sequences:

| | |
|---|---|
| FR1 | EVQLVESGGGLVQA/PGGSLRLS/ACV/AVSGT/PG/-L/-IFS/K |
| CDR1 | Y/FTGVA |
| FR2 | WYRR/-QT/VS/PGK/AQ/KRE/GW/LVA |
| CDR2 | T/LT/ITN/GDGG/ATK/R |
| FR3 | F/YA/GDSVKGRFTI/VSRDN/IAK/AKT/RVYLQ/EMNN/DLN/RA/SEDTAVYYC |
| CDR3 | KTGR/EF/Y |
| FR4 | WGR/GGTL/QVTVSS |

* Inserted amino acid in framework region

An anti-US28 antibody that binds to the extracellular region including, for example, the N-terminal extracellular region and/or extracellular loops of US28, for use in a method of reactivating cytomegalovirus in latently infected cells may be coupled to a cytotoxic drug as effector molecule, preferably as an antibody-drug conjugate (ADC). Said drug conjugate is a chemotherapeutic drug, a photosensitizing chemical, and/or a toxic compound. Said drug conjugate preferably is cleaved internally leading to the release of the drug conjugate in order to induce cell death. Said antibody-drug conjugate is able to kill both lytically infected cells and latently infected cells.

Said chemotherapeutic drug preferably is an alkylating agent such as nitrogen mustard, e.g. cyclophosphamide, mechlorethamine or mustine, uramustine and/or uracil mustard, melphalan, chlorambucil, ifosfamide; nitrosourea, including carmustine, lomustine, streptozocin; an alkyl sulfonate such as busulfan, an ethylenime such as thiotepa and analogues thereof, a hydrazine/triazine such as dacarbazine, altretamine, mitozolomide, temozolomide, altretamine, procarbazine, dacarbazine and temozolomide; an intercalating agent such as a platinum-based compound like cisplatin, carboplatin, nedaplatin, oxaliplatin and satraplatin; anthracyclines such as doxorubicin, daunorubicin, epirubicin and idarubicin; mitomycin-C, dactinomycin, bleomycin, adriamycin, mithramycin and an anti-mitotic drug such as monomethyl auristatin E.

Said photosensitizing chemical preferably is a porphyrin such as aminolevulinic acid (ALA), a chlorin such as tetrahydroxyphenylchlorin (mTHPC), mono-L-aspartyl chlorin E6, and/or a dye such as methylene blue. A preferred photosensitizing chemical is selected from Photofrin (porfimer sodium), Visudyne (verteporfin), Foscan (temoporfin), Allumera, Levulan (aminolevulinic acid), Metvix (methyl aminolevulinate), Cysview (Hexaminolevulinate HCl), and Laserphyrin (talaporfin sodium, which are commercially available. Further photosensitizing chemicals include Tookad (padeliporfin), Antrin (cetirizine dihydrochloride), Photochlor (2-(1-hexyloxyethyl)-2-devinyl pyropheophorbide-a (HPPH) , Photosens, Photrex (ethyl (35,25R)-23,23-dichloro-3,9,14,19-tetraethyl-8,13,18,25-tetramethyl-1,24,26,27-tetraza-23-stannaheptacyclo[10.10.2.13,22.17,10.117,20.02,6.015,24]heptacosa-2(6),4,7(27),8,10,12,14,16,18,20(26),21-undecaene-4-carboxylate, Lumacan (hexaminolevuminate), Cevira (aminolevulinic acid hexyl ester), Visonac (methyl 5-aminolevulinate), BF-200 (5-aminolaevulinic acid), ALA (5-aminolaevulinic acid), Amphinex (tetraphenyl chlorin disulfonate), and azadipyrromethenes are in clinical trials or preclinical development.

Examples of a toxic compound are saporin, bryodin, agrostin, ricin, an exotoxin such as Pseudomonas exotoxin A or a truncated version thereof termed PE38 (Onda et al., 2006. J Immunol 177: 8822-34), diphtheria exotoxin and Vibrio cholerae Cholix toxin, cytolysins such as pneumolysin, perfringolysin and listeriolysin, haemolysin A, an enediyne such as calicheamicin, preferably calicheamicin γ1, and esperamicin, a maytansinoid such as mertansine and emtansine, gelonin, dianthin, luffin, α-momorcharin, β-momorcharin, dodecandrin, tritin, momordin, and trichosanthin.

Said antibody-drug conjugate preferably comprises a stable link between the antibody and the drug conjugate, such as a linker based on a chemical motif such as disulfides, hydrazones, peptides, thioethers, maleimide and/or thiosuccinimide. The anti-US28 antibody, or the anti-US28 antibody-drug conjugate may be used to reactivate cytomegalovirus in latently infected cells, and/or to eliminate latently infected cells, in cells, tissue or an organ that is to be transplanted. For this, said anti-US28 antibody, or anti-US28 antibody-drug conjugate, is preferably provided at a dosage of between 0.5 microgram and 10 milligram per kg per use, which is the incubation of cells that are to be transplanted or the flushing or perfusion of a tissue or organ that is to be transplanted. For example, a lung that is to be transplanted may be perfused ex vivo with a dosage of between 0.5 microgram and 10 milligram per kg, for example with an anti-US28 antibody-photosensitizing chemical, prior to transplantation. Said dosage may be adjusted to provide sufficient levels of the antibody or antibody-conjugate and/or to invoke the desired therapeutic effect. Said tissue, organ and/or cells preferably is/are flushed with an isotonic aqueous solution prior to transplantation in order to eliminate latently infected cells.

As an alternative, or in addition, an anti-US28 antibody that binds to the extracellular region including, for example, the N-terminal extracellular region and/or extracellular loops of US28, and/or an antibody-drug conjugate, may be administered to a seropositive donor and/or a seropositive recipient prior to the transplantation. It has been found that transplant recipients often take immune suppressants, which result in at least a partial reactivation of viruses, including HCMV. In that case, the cytomegalovirus is at least partially reactivated in donor and/or recipient cells. These cells are preferably targeted with an antibody-drug conjugate, for example comprising a photosensitizing chemical, prior to transplantation, in order to kill these cells.

An anti-US28 antibody that binds to the extracellular region including, for example, the N-terminal extracellular region of US28, and/or an anti-US28 antibody-drug conjugate, may be administered to a seropositive donor and/or a seropositive recipient in combination with an antiviral agent and/or a histone deacetylase inhibitor for use in a method of reactivating cytomegalovirus in latently infected cells and/or to eliminate latently infected and/or reactivated cells.

Said antiviral agent preferably is selected from ganciclovir (2-amino-9-(1,3-dihydroxypropan-2-yloxymethyl)-1H-purin-6-one; preferably by intravenous injection of 1-10 mg, preferably about 5 mg per kilogram of body weight once or twice per day for at least 7 days); valganciclovir ([2-[(2-amino-6-oxo-1H-purin-9-yl)methoxy]-3-hydroxypropyl](2S)-2-amino-3-methylbutanoate; preferably by oral provision of 500-2000, preferably about 900 mg once or twice per day for at least 7 days); foscarnet (phosphonoformic acid; preferably by intravenous injection or infusion of 50-500 mg, preferably about 90 mg per kilogram of body weight once or twice per day for at least 7 days); cidofovir ([(2S)-1-(4-amino-2-oxopyrimidin-1-yl)-3-hydroxypropan-2-yl]oxymethylphosphonic acid; preferably by intravenous injection or infusion of 1-10 mg, preferably about 5 mg per kilogram of body weight once or twice per week for at least 2 weeks); letermovir (2-[(4S)-8-fluoro-2-[4-(3-methoxyphenyl)piperazin-1-yl]-3-[2-methoxy-5-(trifluoromethyl)phenyl]-4H-quinazolin-4-yl]acetic acid; preferably by injection or oral provision of 200-800, mg. preferably about 480 mg once or twice per day for at least 7 days); maribavir ((2S,3S,4R,5S)-2-[5,6-dichloro-2-(propan-2-ylamino)benzimidazol-1-yl]-5-(hydroxymethyl)oxolane-3,4-diol; preferably by oral provision of 200-1200 mg. preferably about 700 mg once or twice per day for at least 7 days); or a combination thereof.

Said histone deacetylase inhibitor preferably is selected from a hydroxamic acid such as trichostatin A ((2E,4E,6R)-7-[4-(dimethylamino)phenyl]-N-hydroxy-4,6-dimethyl-7-oxohepta-2,4-dienamide), (E)-N-hydroxy-3-[3-(phenylsulfamoyl)phenyl]prop-2-enamide) and suberoylanilide hydroxamic acid (N′-hydroxy-N-phenyloctanediamide), an aliphatic acid such as butanoic acid, valproic acid, phenylbutyrate and 4-phenylbutanoic acid, a cyclic peptide such as a depsipeptide such as romidepsin ((1S,4S,7Z,10S,16E,21R)-7-ethylidene-4,21-di(propan-2-yl)-2-oxa-12,13-dithia-5,8,20,23-tetrazabicyclo[8.7.6]tricos-16-ene-3,6,9,19,22-pentone) and apicidin ((3S,6S,9S,12R)-3-[(2S)-butan-2-yl]-6-[(1-methoxyindol-3-yl)methyl]-9-(6-oxooctyl)-1,4,7,10-tetrazabicyclo[10.4.0]hexadecane-2,5,8,11-tetrone), a benzamides such as entinostat (pyridin-3-ylmethyl N-[[4-[(2-aminophenyl)carbamoyl]phenyl]methyl]carbamate), mocetinostat (N-(2-aminophenyl)-4-[[(4-pyridin-3-ylpyrimidin-2-yl)amino]methyl]benzamide) and tacedinaline (4-acetamido-N-(2-aminophenyl)benzamide).

Factors that can be taken into account when determining a dosage of the anti-US28 antibody or anti-US28 antibody-drug conjugate, in combination with an antiviral agent and/or a histone deacetylase inhibitor, include the general health of the subject, age, weight and gender of the subject, diet, time and frequency of administration, drug combination(s), reaction sensitivities and tolerance/response to therapy, as is known to a person skilled in the art.

As an alternative, the antibody-drug conjugate may be administered after starting transplantation, or continued after starting or completion of a transplantation, to the transplantation recipient.

In addition, the anti-US28 antibody, or anti-US28 antibody-drug conjugate, either alone or in combination with an antiviral agent and/or a histone deacetylase inhibitor, may be administered to a HCMV-infected woman in a preconceptional phase, a pregnant women, for example a pregnant women who is infected with HIV or who uses immunosuppressants, a fetus that is infected in utero, a neonate, who is immune-naive and at risk of a primary infection, a lactating mother to clear the latent reservoir from the mammary glands to prevent transmission, a HIV patient to prevent amelioration in case of low CD4+ and CD8+ count, an otherwise immunodeficient individual, a cancer patient who is receiving chemotherapy, whereby the anti-US28 antibody, or anti-US28 antibody-drug conjugate, either alone or in combination with an antiviral agent and/or a histone deacetylase inhibitor may be administered before, during or after chemotherapy.

Said anti-US28 antibody, either alone or in combination with an antiviral agent and/or a histone deacetylase inhibitor, and said anti-US28 antibody-drug conjugate, is preferably provided as a pharmaceutical composition. Said pharmaceutical composition preferably is a sterile isotonic solution. Said buffer preferably is citrate-based buffer, preferably lithium-, sodium-, potassium-, or calcium- citrate monohydrate, citrate trihydrate, citrate tetrahydrate, citrate pentahydrate, or citrate heptahydrate; lithium, sodium, potassium, or calcium lactate; lithium, sodium, potassium, or calcium phosphate; lithium, sodium, potassium, or calcium maleate; lithium, sodium, potassium, or calcium tartarate; lithium, sodium, potassium, or calcium succinate; or lithium, sodium, potassium, or calcium acetate, or a combination of two or more of the above. The pH of said buffer may be adjusted, preferably to a pH of 7.27-7.37 by hydrochloric acid, sodium hydroxide, citric acid, phosphoric acid, lactic acid, tartaric acid, succinic acid, or a combination of two or more of the above. The volume of may range from 0.5 ml to 5 ml. Said excipient preferably is selected from, but not limited to, urea, L-histidine, L-threonine, L-asparagine, L-serine, L-glutamine, polysorbate, polyethylene glycol, propylene glycol, polypropylene glycol, or a combination of two or more of the above.

Said pharmaceutical composition preferably comprises a kit of parts, comprising a pharmaceutical preparation comprising the anti-US28 antibody and a pharmaceutical preparation comprising the antiviral agent and/or HDAC inhibitor. Said kit of parts preferably further comprising instructions for administration of the anti-US28 antibody prior to the administration of the antiviral agent and/or the HDAC inhibitor, preferably one day, preferably two days, preferably 3 days, preferably more than 3 days such as 1-2 weeks prior to the administration of the antiviral agent.

A further preferred pharmaceutical composition comprises liposomes, said liposomes comprising the anti-US28 antibody. Said liposomes preferably further comprise an antiviral agent and/or the HDAC inhibitor.

A pharmaceutical composition according to the invention preferably is for use in a method of activating HCMV in a tissue, organ or cells that are or will be transplanted.

### EXAMPLES

### Example 1

### Materials and Methods

### Cell Culture and Virus Infection

Viral isolate RV1164 (HCMV TB40/E strain with an IE2-YFP tag) has been described previously (Weekes et al., 2013. Science 340: 199-202). Primary CD14+ monocytes were isolated from apheresis cones (NHS Blood and Transfusion Service, United Kingdom), as described previously (Poole et al., 2014. Methods Mol Biol 1119: 81-98), or by MACS separation using CD14 microbeads (Miltenyi, United Kingdom) as described previously (Poole et al., 2013. J Virol 87: 4261-71). CD14+ monocytes were cultured in X-vivo 15 (Lonza; Walkersville, Md.) at 37° C. in 5% CO 2. Cells were infected with at a multiplicity of infection (MOI) of 3 (based on infection of RPE-1 cells). THP-1 cells, lentivirally transduced with different US28 constructs, were described earlier (Krishna et al., 2017. MBio 8: 1-21) and were cultured according to ATCC standards.

### Immunofluorescence Microscopy on THP-1 Cells

THP-1 cells were spun down at 50033 g for 5 min, resuspended in 4% paraformaldehyde (Sigma-Aldrich) and seeded in a 96 well U-bottom plate. Cells were fixed for 10 minutes at room temperature. After fixation, cells were permeabilized with 0.5% NP-40 (Sigma-Aldrich) for 30 min at room temperature. Nanobodies were incubated for 1 h at RT and detected using Mouse-anti-Myc antibody (1:1000, 9B11 clone, Cell Signaling). US28 was visualized with the rabbit-anti-US28 antibody (1:1000, Covance (Slinger et al., 2010. Sci Signal 3: ra58). Subsequently, cells were washed and incubated with Goat-anti-Rabbit Alexa Fluor 546 (1:1000 in 1% (v/v) FBS /PBS, Thermo Fisher Scientific) and Goat-anti-Mouse Alexa Fluor 488 (1:1000 in 1% (v/v) FBS/PBS, Thermo Fisher Scientific).

### Detection of Immediate Early Expression

CD14+ monocytes were isolated and seeded in a 96 wells plate. As a positive control, CD14+ monocytes were pre-treated with 20 ng/ml phorbol myristate acetate (PMA) one day after seeding. The next day, medium was removed and cells were infected with RV1164 viral isolate. Two hours post infection, medium was aspirated and replaced with medium containing nanobodies (final concentration of 100 nM). Staining of immediate early 2 days post infection was done as described earlier [15]. 3 days post infection, nanobody-containing medium was refreshed. 6 days post infection, YFP-positive cells were counted.

### RT-PCR

CD14+ monocytes were isolated and infected as described above. 6 days post infection, cells were washed once with 1×PBS and RNA was harvested by adding Trizol reagent (Life technologies). RNA was isolated using miRNeasy mini kit (Qiagen) according to manufacturer's instructions. cDNA was produced by Quantitect Reverse Transcription kit (Qiagen) according to manufacturer's instructions. RT-PCR was performed as described previously (Krishna et al., 2016. Sci Rep 6: 24674). Gene expression was calculated using ΔΔct method and normalized to GAPDH gene expression.

### T Cell Co-Culture

The T cell co-culture was performed as described previously (Krishna et al., 2016. Sci Rep 6: 24674). Briefly, CD14+ monocytes and peripheral blood mononuclear cells (PBMCs) of HCMV-positive donor were isolated, infected with RV1164 viral isolate and treated with nanobodies as described above. Six days post infection, the T cells or T-cell depleted PBMCs of the donor were co-cultured with the CD14+ monocytes for 2 days. T-cells or PBMCs were removed and CD14+ monocytes were differentiated to immature dendritic cells by addition of interleukin-4 and by granulocyte-macrophage colony-stimulating factor at 1,000 U/ml for 5 days. Next, lipopolysaccharide at 500 ng/ml was added for 2 days to induce mature dendritic cells.

### RESULTS

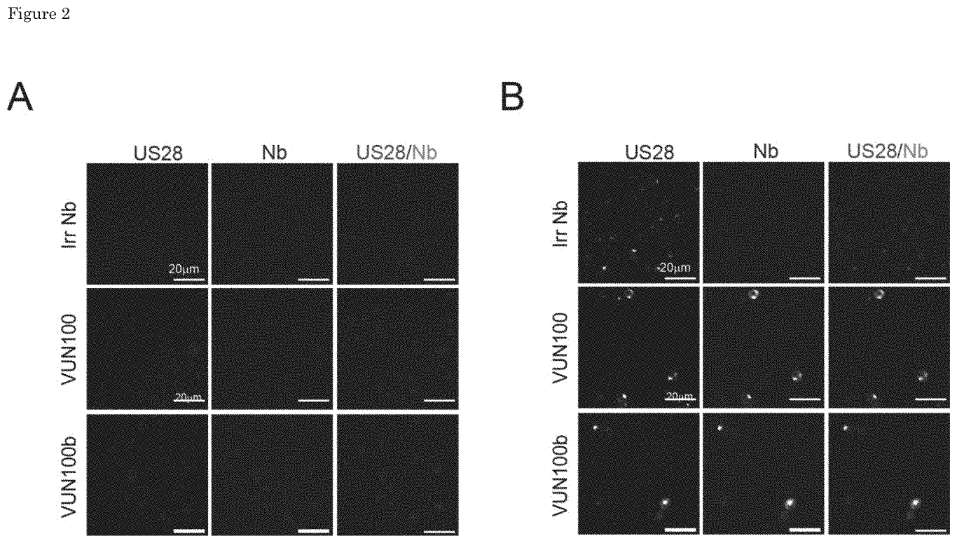

Binding of monovalent VUN100, bivalent VUN100b, or irrelevant nanobody were tested on both US28-expressing and mock transduced THP-1 cells (FIG. 2). VUN100 and VUN100b showed specific binding to US28-expressing THP-1 cells while this was not seen for the irrelevant nanobody (FIG. 2B). In contrast, no binding of all three nanobodies to the mock transduced THP-1 cells (FIG. 2A).

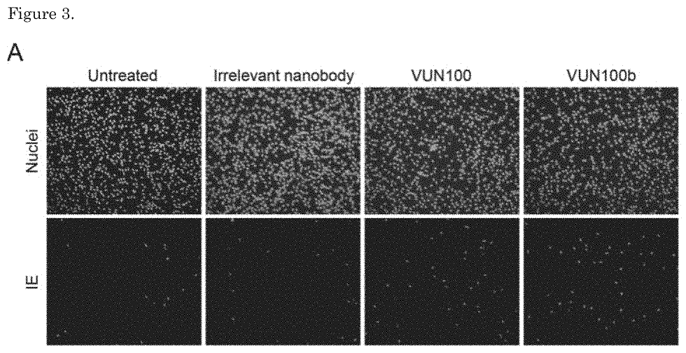

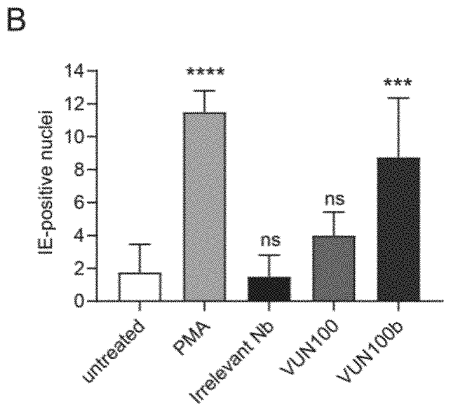

To assess the effect of the single heavy chain variable domain antibodies on immediate early (IE) expression, primary CD14+ monocytes were infected with the viral HCMV isolate RV1164, which has an IE2-YFP tag, and treated with yjr antibodies. Two days post infection, cells were fixed and stained for IE-expression (FIG. 3A). Treatment of the cells with US28 nanobodies resulted in an increase of IE2-expression while this was not seen for the cells treated with the irrelevant nanobody. Six days post infection, IE2-positive cells were counted (FIG. 3B). VUN100b treatment resulted in a significant increase of IE2-expressing CD14+00 monocytes. Monovalent VUN100 also showed an increase although not significant. These results show that predominantly VUN100b induced IE expression which indicates the first steps towards reactivation of latently infected cells. As a control of reactivation, cells were pre-treated with phorbol myristate acetate (PMA) to induce differentiation of CD14+ monocytes. This resulted in a significant increase of IE2-positive cells compared to untreated CD14+ monocytes. To determine whether antibody treatment resulted in full reactivation, CD14+ monocytes were co-cultured with fibroblasts as a method to detect actual virus production. Interestingly, no viral plaques were observed after VUN100b treatment, indicating that antibody treatment resulted in an increase of IE-expression but no full reactivation (not shown).

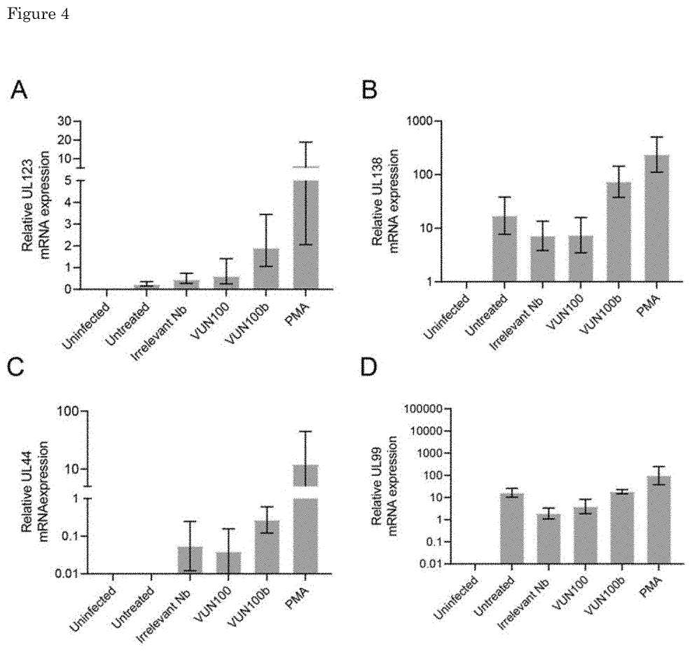

Since VUN100b treatment did not result in full viral reactivation, different gene expression markers were tested upon antibody treatment of HCMV-infected CD14+ monocytes (FIG. 4). VUN100b treatment of HCMV-infected cells resulted in an increase of UL123 (FIG. 4A), UL138 (FIG. 4B) and UL44 (FIG. 4C) gene expression, but not of the true late gene UL99 (FIG. 4D) supporting our findings that VUN100b treatment do not result in full reactivation.

Since HCMV-positive donors have a high frequency of HCMV-specific cytotoxic T cells, we evaluated whether the partial reactivation of infected CD14+ monocytes by VUN100b would be sufficient to result in clearance of these cells by cytotoxic T cells.

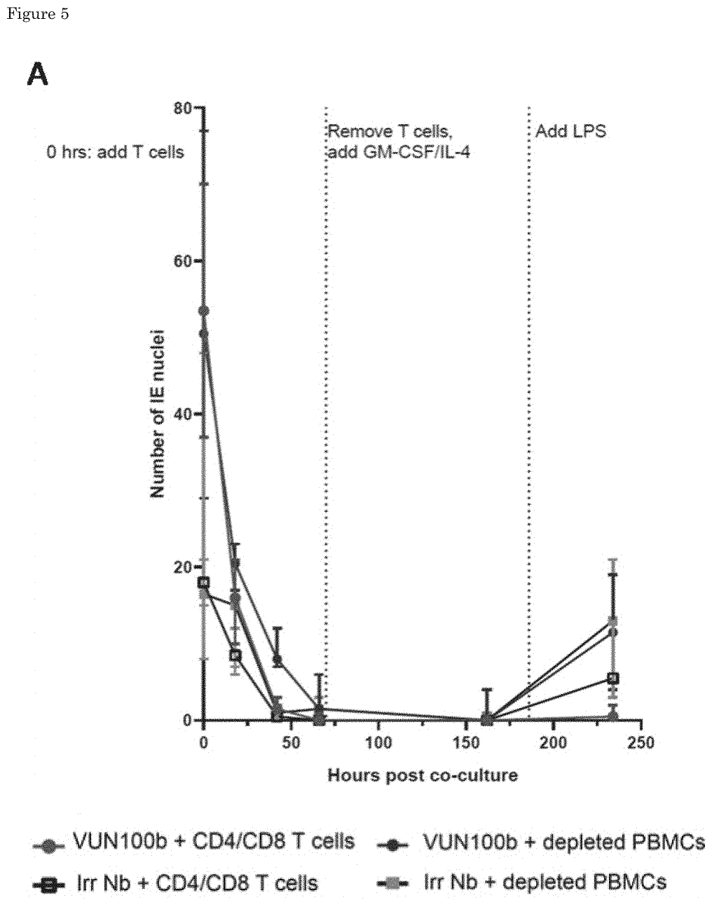

For this. CD14+ monocytes, T cells and T cell-depleted PBMCs were isolated of HCMV-positive donors. Six days post infection and antibody treatment, IE-positive cells were counted (FIG. 5B). As seen previously, VUN100b treatment or pretreatment of the cells with PMA resulted in an increase of IE expression compared to CD14+ monocytes which were treated with an irrelevant antibody. Monocytes were co-cultured with T cells or T cell depleted PBMCs (FIG. 5A). Importantly, no significant differences in IE-expression was seen between the wells of each treatment before co-culturing (FIG. 5B). After 48 hours of co-culturing, T cells were removed and CD14+ monocytes were differentiated into mature dendritic cells to fully reactivate the remaining HCMV-infected CD14+ monocytes (FIG. 5C). Interestingly, after T cell co-culturing, almost no IE-positive CD14+ monocytes were observed upon treatment with either VUN100b, or with positive control PMA, while this was not seen after co-culturing with T cell depleted PBMCs. Treating with irrelevant Nb did not decrease the number of IE-positive CD14+ monocytes significantly upon co-culturing with T cells compared to the co-culture with T cell depleted PBMCs. These data indicate that besides full reactivation by PMA, partial reactivation of latently infected cells by VUN100b is already sufficient to induce IE expression and allow specific T cell-mediated removal of latently infected monocytes.

### EXAMPLE 2

### Materials and Methods

See Example 1.

### RESULTS

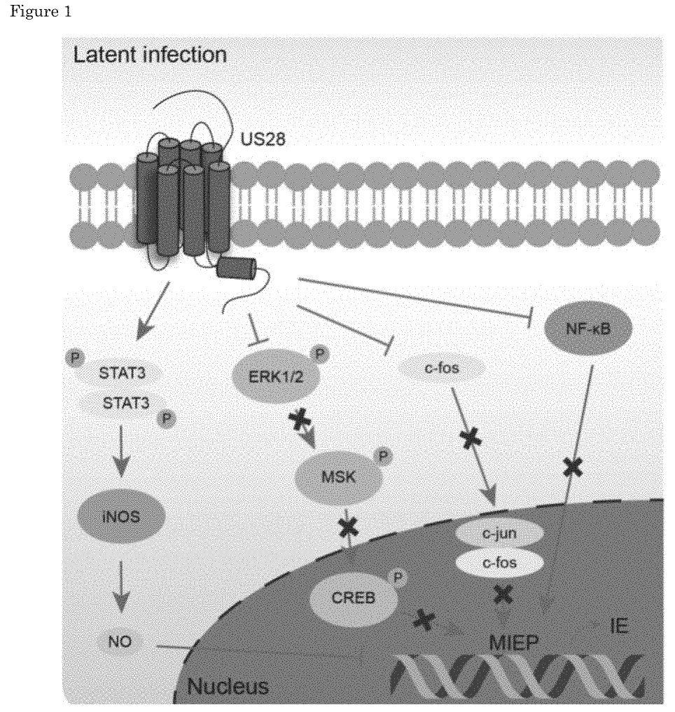

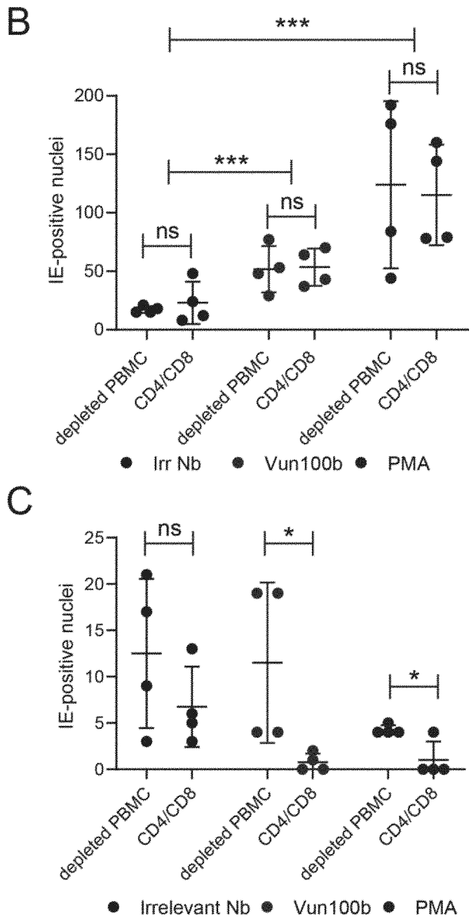

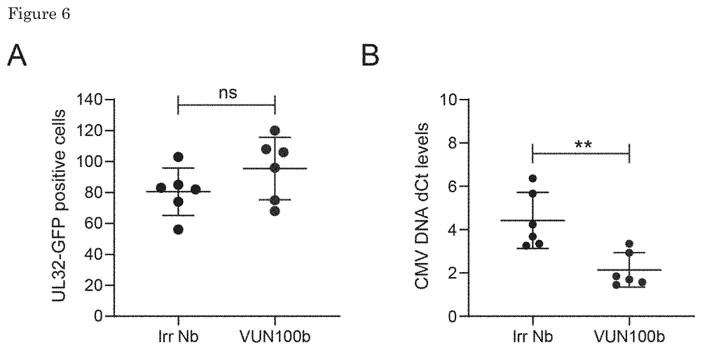

A drawback of the HCMV IE2-YFP virus used in the analysis depicted in Example 1 is that it contains a deletion of the virus US2-US6 region. This region encodes several proteins that interfere with antigen presentation by, for example, downregulating MHC Class I and II molecules (Noriega et al., 2012. Immunol Res 54: 140-151; Wiertz et al., 1996. Cell 84: 769-779; Johnson and Hegde, 2002. Curr Top Microbiol Immunol 269: 101-115). Though it should be pointed out, that this HCMV IE2-YFP virus does encode US11 (as shown by RT-qPCR, data not shown), which can downregulate some MHC Class I molecules (Zimmermann et al., 2019. PLoS Pathog 15: e1008040; Ameres et al., 2014. J Immunol 192: 5894-5905). However, to ensure that our observations would also be recapitulated with a virus with a full complement of immune evasins, we repeated the co-culture experiments described in Example 1 with a HCMV strain containing an intact US2-6 region. For visualisation and quantification of infection, and in contrast to the IE2-YFP, this virus expresses a C-terminally GFP tagged UL32 protein (FIG. 6; Sampaio et al., 2005. J Virol 79: 2754-2767). CD14+ monocytes from HCMV-positive donors were infected with HCMV UL32-GFP-and treated with VUN100b or an irrelevant nanobody. Six days post infection and nanobody treatment, GFP-positive cells were counted (FIG. 6A). In line with our observations showing that VUN100b induces IE gene expression but does not induce late lytic gene expression, no significant difference in the number of UL32-GFP positive cells was observed between the cells treated with the irrelevant nanobody or VUN100b. Upon 48 hours of co-culturing of the infected CD14+ monocytes with T cells from HCMV positive donors, T cells were removed, all CD14+ monocytes were differentiated into mature dendritic cells. To assess viral load, these dendritic cells were co-cultured with Hff1 fibroblast cells (ATCC No: SCRC-1041). After 7 days of co-culturing HCMV DNA genome levels were determined (FIG. 1B). In line with the results obtained with the IE2-YFP virus, a significant reduction (P value=0.0043) of HCMV DNA levels was observed for the VUN100b-treated CD14+ monocytes compared to the monocytes treated with the irrelevant nanobody. Overall, these data indicate that partial reactivation of latently infected cells by VUN100b is sufficient to induce IE-expression to levels that allow HCMV-specific T cell-mediated clearing of latently infected monocytes.

### EXAMPLE 3

### Materials and Methods

See Example 1.

### RESULTS

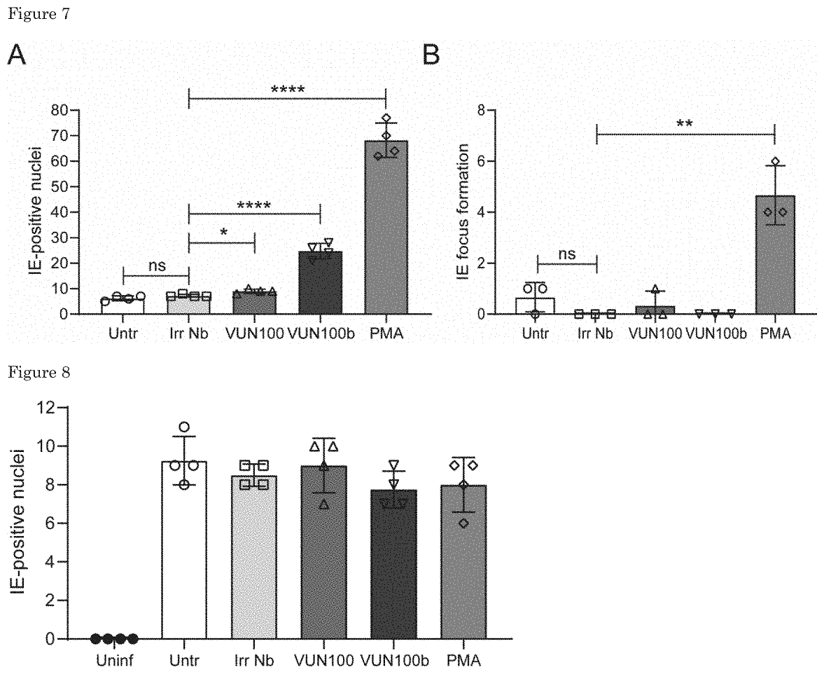

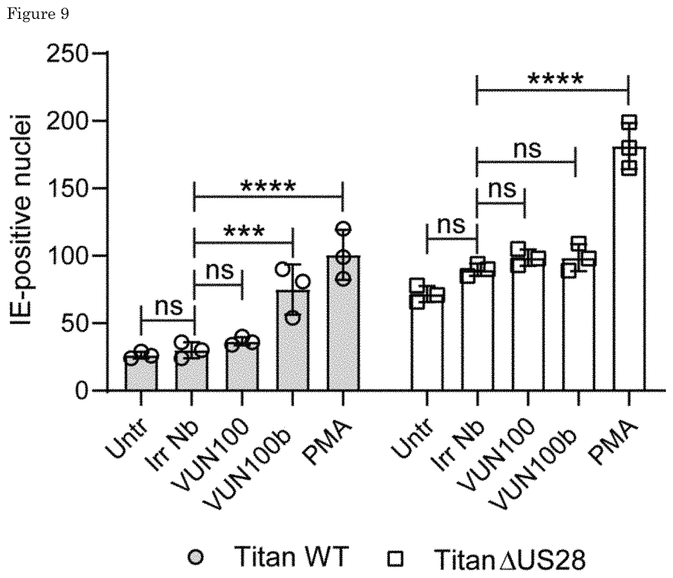

To ensure that VUN100b reverses latency and not only prevents the establishment of latency by US28, the nanobodies or PMA were added after establishment of latency (six days post infection, FIG. 7A) instead of 2 hours post infection. Six days post infection and before treatment, no significant differences in IE-expression between the different wells was observed (FIG. 8). Again, VUN100b treatment resulted in a significant upregulation of IE-expression (P value<0.0001) compared to the untreated or irrelevant nanobody treated monocytes. Treatment with monovalent VUN100 also resulted in a very small but significant upregulation of IE-expression (P value=0.01). Moreover, as seen previously, no production of infectious viral particles was observed from the untreated and nanobody-treated monocytes upon co-culturing with fibroblasts for eight days (FIG. 7B). In contrast, co-culturing of PMA-treated monocytes with fibroblasts resulted in a significant upregulation of IE-focus formation (P value=0.0022). Finally, to ensure that the effect of VUN100b is US28 specific, we performed the same experiments with Titan wildtype (WT) and Titan AUS28 virus (FIG. 9). Also in this setting, using the Titan WT virus, we noticed a significant upregulation of IE-expression upon VUN100b (P value=0.0003) or PMA treatment (P value<0.0001) of latently infected cells compared to the untreated or irrelevant nanobody treated cells. In contrast, using the Titan-AUS28 virus, increased of IE-expression was observed, caused by the lack of US28 expression by this virus. However, IE-expression could be further increased by PMA-treatment (P value<0.0001). Treatment of the Titan-AUS28 infected CD14+ monocytes with the irrelevant nanobody resulted in a small, but not significant, increase of IE-expression compared to the untreated cells, which could be due to a non-specific effect of the irrelevant nanobody, residual contaminants or the sheer manipulation of the cells. However, most importantly, VUN100b treatment did not result in higher IE-expression compared to the irrelevant nanobody, indicating that the effect of VUN100b is US28-specific.

1-15. (canceled)

16. A method of reactivating cytomegalovirus in infected cells, comprising incubating cells with a single heavy chain variable domain antibody against human cytomegalovirus protein US28, which antibody binds to the extracellular region of US28, to thereby reactivate cytomegalovirus in said infected cells.

17. The method of claim 16, wherein said antibody binds to the N-terminal extracellular region and/or the extracellular loops of US28.

18. The method according to claim 16, wherein said method is an ex vivo method.

19. The method according to claim 16, wherein said antibody comprises complementarity-determining regions (CDRs) having amino acid sequences (F/Y)TGVA for CDR1; (L/T/S)(I/T/A)T(G/N)DG(A/G)T(R/K) for CDR2; and KTG(E/R)(Y/F) for CDR3.

20. The method according to claim 16, wherein said antibody comprises human or humanized framework regions.

21. The method according to claim 16, wherein said antibody is fused to an immunoglobulin Fc region or functional part thereof.

22. The method according to claim 21, wherein the Fc region or functional part thereof is from or derived from, IgG1, IgG2, IgG3, or IgG4.

23. The method according to claim 21, wherein said antibody is fused to a human or humanized Fc region, or functional part thereof.

24. The method according to claim 21, wherein said antibody is part of a bi- or multivalent antibody.

25. The method according to claim 16, wherein said infected cells are present in an individual.

26. The method according to claim 16, wherein said infected cells are present in an organ that is to be transplanted.

27. The method according to claim 16, wherein said infected cells are present in stem cells that are to be transplanted.

28. The method according to claim 27, wherein said infected cells are bone marrow stem cells.

29. The method according to claim 16, wherein said method further comprises administering an anti-viral agent and/or a histone deacetylase inhibitor.

30. The method according to claim 16, wherein said infected cells are latently infected with human cytomegalovirus (HCMV), or cells in which HCMV is at least partially reactivated.

31. The method according to claim 16, wherein said antibody is coupled to a cytotoxic drug and/or a photosensitizer.

32. A tissue, organ, or cells, from which cells that were infected with CMV have been removed by incubating the cells with a single heavy chain variable domain antibody against human cytomegalovirus protein US28 that binds to the extracellular region of US28, to thereby reactivate cytomegalovirus in said infected cells.

33. The tissue, organ, or cells according to claim 32, comprising bone marrow stem cells.

34. A method of identifying cells that are infected with cytomegalovirus, comprising incubating cells with a single heavy chain variable domain antibody against human cytomegalovirus protein US28, which antibody binds to the extracellular region of US28, to thereby identify said infected cells.

35. The method of claim 34, wherein said antibody binds to the N-terminal extracellular region and/or the extracellular loops of US28.

36. The method of claim 34, whereby said antibody is coupled to a tag.

37. A method of isolating cells that are infected with cytomegalovirus, comprising incubating cells with a single heavy chain variable domain antibody against human cytomegalovirus protein US28, which antibody binds to the extracellular region of US28, and isolating cells that are bound to said single heavy chain variable domain antibody.

38. The method of claim 37, wherein said antibody binds to the N-terminal extracellular region and/or the extracellular loops of US28.

39. The method of claim 37, whereby the antibody is coupled to a tag.
